(19) United States
(12) Patent Application Publication
(10) Pub. No.: US 2015/0203851 A1
(43) Pub. Date: Jul. 23, 2015

(54) INHIBITORS OF BROMODOMAIN-CONTAINING PROTEIN PCAF FOR THE TREATMENT OF AUTOIMMUNE AND INFLAMMATORY DISEASES OR FOR THE TREATMENT OF CANCER

(71) Applicant: Glaxo Group Limited, Brentford, Middlesex (GB)

(72) Inventors: Laurens Kruidenier, Stevenage (GB); Kevin Lee, Stevenage (GB); David Francis Tough, Stevenage (GB); David Matthew Wilson, Stevenage (GB)

(21) Appl. No.: 14/425,061

(22) PCT Filed: Sep. 4, 2013
(86) PCT No.: PCT/EP2013/068219

§ 371 Date: Mar. 2, 2015

(30) Foreign Application Priority Data
Sep. 6, 2012 (GB) 1215942.2

(51) Int. Cl. C12N 15/113; G01N 33/50

(52) U.S. Cl. CPC C12N 15/1137; G01N 33/5047; G01N 33/502; C12N 2320/30; C12N 2310/14; G01N 2333/91051

(57) ABSTRACT

A method of treating autoimmune and inflammatory diseases or conditions or cancer in a mammal, such as a human, which comprises the administration of an inhibitor of the bromodomain-containing protein PCAF.

### FIELD OF THE INVENTION

The present invention is concerned with new methods of treatment. More particularly, the present invention relates to methods for treatment or prevention of autoimmune and inflammatory diseases and conditions and cancer by inhibiting or modifying the expression or function of bromodomain-containing proteins. In a further aspect the invention relates to a method for identifying agents useful in said methods of treatment. The invention particularly describes the role of certain bromodomain-containing proteins, particularly p300/CBP-associated factor (PCAF, also known as K(lysine)acetyltransferase 2B (KAT2B)) in these diseases and conditions and their use as therapeutic and screening targets.

### BACKGROUND OF THE INVENTION

Chromatin is the complex combination of DNA and protein that makes up chromosomes. It is found inside the nuclei of eukaryotic cells and is divided between heterochromatin (condensed) and euchromatin (extended) forms. A range of different states of condensation are possible and the tightness of this structure varies during the cell cycle, being the most compact during the process of cell division. The major components of chromatin are DNA and proteins. Histones are the chief protein components of chromatin, acting as spools around which DNA winds. The basic building blocks of chromatin are nucleosomes, each of which is composed of 146 base pairs of DNA wrapped around a histone octamer that consists of 2 copies of each H2A, H2B, H3 and H4. The functions of chromatin are to package DNA into a smaller volume to fit in the cell, to strengthen the DNA to allow mitosis and meiosis, and to serve as a mechanism to control expression and DNA replication. Chromatin contains genetic material serving as instructions to direct cell functions. The genomes of eukaryotic organisms are highly organised within the nucleus of the cell. The chromatin structure is controlled by a series of post translational modifications to histone proteins, notably histones H3 and H4, and most commonly within the “histone tails” which extend beyond the core nuclerosome structure. Histone tails tend to be free for protein—protein interaction and are also the portion of the histone most prone to post-translational modification. These modifications include acetylation, methylation, phosphorylation, ubiquitinylation, SUMOylation. These epigenetic marks are written and erased by specific enzymes, which place the tags on specific residues within the histone tail, thereby forming an epigenetic code, which is then interpreted by the cell to allow gene specific regulation of chromatin structure and thereby transcription.

Of all classes of proteins, histones are amongst the most susceptible to post-translational modification. Histone modifications are dynamic, as they can be added or removed in response to specific stimuli, and these modifications direct both structural changes to chromatin and alterations in gene transcription. Lysine acetylation is a histone modification that forms an epigenetic mark on chromatin for bromodomain-containing proteins to dock and in turn, regulate gene expression. Distinct classes of enzymes, namely histone acetyltransferases (HATs) and histone deacetylases (HDACs), acetylate or de-acetylate specific histone lysine residues (1).

The bromodomain is currently the only protein domain known to bind specifically to acetylated lysine residues in histone tails. Bromodomains, which are approximately 110 amino acids long, are found in a large number of chromatin-associated proteins and have now been identified in approximately 70 human proteins, often adjacent to other protein motifs (2,3). Proteins that contain a bromodomain may contain additional bromodomains, as well as other functional motifs. For example, many HATs also contain a bromodomain (2). Interactions between bromodomains and modified histones may be an important mechanism underlying chromatin structural changes and gene regulation. Bromodomain-containing proteins have been implicated in disease processes including cancer, inflammation and viral replication. The development of inhibitors to bromodomains is thus an attractive means for controlling gene expression, and there is a need in the art to regulate bromodomain binding to acetylated lysine in order to control gene expression.

The present inventors have identified bromodomain-containing proteins involved in the inflammatory response. Inhibiting these bromodomain-containing proteins by inhibiting expression and/or function therefore would provide a novel approach to the treatment of autoimmune and inflammatory diseases or conditions or cancer.

### SUMMARY OF THE INVENTION

Thus, in one aspect there is provided a method of treating autoimmune and inflammatory diseases and conditions or cancer which comprises inhibiting the bromodomain-containing protein PCAF in a mammal.

In a further aspect there is provided a method of treatment of autoimmune and inflammatory diseases or conditions or cancer in a mammal comprising administering a therapeutically effective amount of an inhibitor of the bromodomain-containing protein PCAF.

In a further aspect there is provided the use of an inhibitor of the bromodomain-containing protein PCAF in the manufacture of a medicament for the treatment of autoimmune and inflammatory diseases and conditions or cancer in a mammal.

In a further aspect the present invention provides an inhibitor of the bromodomain-containing protein PCAF for use in the treatment of autoimmune and inflammatory diseases and conditions or cancer.

In a further aspect there is provided a pharmaceutical formulation for use in the treatment of autoimmune and inflammatory diseases or conditions or cancer, comprising an inhibitor of the bromodomain-containing protein PCAF, together with at least one pharmaceutical carrier.

In a further aspect, there is provided a method of screening for an inhibitor of the bromodomain-containing protein PCAF, in particular comprising the step of determining whether the compound inhibits or the step of determining whether the compound activates the bromodomain-containing protein PCAF.

### DESCRIPTION OF DRAWINGS

FIG. 1. siRNAs targeting PCAF inhibit inflammatory cytokine production by human dendritic cells. Human monocyte-derived dendritic cells were transfected with siRNAs targeting 40 different bromodomain containing proteins (4 siRNAs per protein), with a scrambled non-targeting siRNA as a negative control, or with an siRNA targeting TNF-α as a positive control. Transfected cells were stimulated with lipopolysaccharide (LPS) and the quantities of cytokines present in the medium approximately 24 hours later were measured. The data show the percent inhibition of TNF-α, as compared to the mean for all siRNA samples on the plate (not including TNF-α siRNA positive control wells), by two different siRNAs targeting PCAF in dendritic cells from 4 separate donors.

FIG. 2. siRNAs targeting PCAF inhibit TNF-α and IL-12 production by human dendritic cells. Monocyte-derived dendritic cells were transfected with two different siRNAs targeting PCAF or with a scrambled non-targeting siRNA as a negative control. The transfected cells were stimulated with LPS and the quantities of cytokines present in the medium 24 hours later were measured. The results are plotted as a fraction of the amount of cytokine produced by PCAF siRNA-transfected cells compared to that produced in control siRNA-transfected cells, with the data normalised to account for possible differences in cell viability (i.e., values=(cytokine amount in sample/cytokine amount in control)/(viability in sample/viability in control)). The data show the mean (+standard deviation) for 9 (PCAF_{—}9), 6 (PCAF_{—}6, TN F-α) or 5 (PCAF_{—}6, IL-12p70) donors.

FIG. 3. Dendritic cells from PCAF KO mice produce lower amounts of pro-inflammatory cytokines than WT dendritic cells in response to multiple stimuli. Bone marrow-derived dendritic cells from WT mice (solid bars) or PCAF KO mice (open bars) were treated with the indicated stimuli and the quantities of cytokines present in the medium 24 hours later were measured. Graphs on the left hand side (A,C,E,G,I,K) show the results when using one concentration of each stimulus (100 ng/ml LPS, 10 μM resiquimod, 10 μM ODN2216, 40 μM imiquimod, 40 μM CL264), while those on the right had side (B,D,F,H,J,L) show the results with a lower concentration of each stimulus (10 ng/ml LPS, 5 μM resiquimod, 5 μM ODN2216, 20 μM imiquimod, 20 μM CL264). Measured cytokines are indicated on the left hand side. The data represent mean values for cells from 3 mice (+/−SEM), except for ODN2216 stimulation, where the results represent mean values from 2 mice (+/−SD). Very similar inhibition was observed when cytokines were measured 6 hours after stimulation (not shown).

FIG. 4. Macrophages cells from PCAF KO mice produce lower amounts of pro-inflammatory cytokines than WT macrophages in response to multiple stimuli. Bone marrow-derived macrophages from WT mice (solid bars) or PCAF KO mice (open bars) were treated with the indicated concentrations of LPS, and the quantities of cytokines present in the medium 6 hours later were measured. The data represent mean values for cells from 7-9 mice (+/−SEM).

### DETAILED DESCRIPTION OF THE INVENTION

Various bromodomain-containing proteins have been identified and characterised. The following is particularly mentioned:

PCAF:

The nucleic acid sequence of human PCAF mRNA is provided by the accession number NM_{—}003884.4.

The amino acid sequence of human PCAF protein is provided by the accession number: NP_{—}003875.3

CBP and p300 are large nuclear proteins that bind to many sequence-specific factors involved in cell growth and/or differentiation. The protein encoded by the PCAF gene associates with p300/CBP; it has in vitro and in vivo binding activity with CBP and p300. PCAF has histone acetyltransferase activity with core histone and nucleosome core particles indicating that this protein plays a role in transcriptional regulation.

As used herein the term “polypeptide” refers to an amino acid chain including a full-length protein, oligopeptides, short peptides and fragments thereof, wherein the amino acid residues are linked by covalent bonds.

As used herein a “variant” is a polypeptide comprising a sequence, which differs (by deletion of an amino acid, insertion of an amino acid, and/or substitution of an amino acid for a different amino acid) in one or more amino acid positions from that of a parent polypeptide sequence. The variant sequence may be a non-naturally occurring sequence, i.e. a sequence not found in nature.

As used herein the term “synthetic peptide” refers to a peptide, including a short peptide that has been synthesized in vitro. The term further encompasses peptides or short peptides that have been modified by substitution with unusual or non-natural amino acids.

As used herein “naturally occurring” as applied to an object refers to the fact that the object can be found in nature as distinct from being artificially produced by man.

As used here in a “fragment” or “subsequence” refers to any portion of a given sequence. It is to be understood that a fragment or subsequence of a sequence will be shorter than the sequence itself by at least one amino acid or one nucleic acid residue. Thus, a fragment or subsequence refers to a sequence of amino acids or nucleic acids that comprises a part of a longer sequence of amino acids (e.g. polypeptide).

As used herein the term “sequence identity” indicates a quantitative measure of the degree of homology between two amino acid sequences or between two nucleic acid sequences of equal length. If the two sequences to be compared are not of equal length, they must be aligned to give the best possible fit, allowing the insertion of gaps or, alternatively, truncation at the ends of polypeptide sequences or nucleotide sequences.

As used herein the term “nucleic acid molecule” refers to an oligomer or polymer of ribonucleic acid (RNA) or deoxyribonucleic acid (DNA) or mimetics thereof. This term includes molecules composed of naturally-occurring nucleobases, sugars and covalent internucleoside (backside) linkages which function similarly or combinations thereof.

As used herein a “polynucleotide sequence” (e.g. a nucleic acid, polynucleotide, oligonucleotide, etc.) is a polymer of nucleotides comprising nucleotides A,C,T,U,G, or other naturally occurring nucleotides or artificial nucleotide analogues, or a character string representing a nucleic acid, depending on context. Either the given nucleic acid or the complementary nucleic acid can be determined from any specified polynucleotide sequence.

As used herein, the term “inhibitor ” can be any compound or treatment capable of inhibiting the expression and/or function of the bromodomain- containing protein, i.e. any compound or treatment that inhibits transcription of the gene, RNA maturation, RNA translation, post-translational modification of the protein, binding of the protein to an acetylated lysine target and the like. Thus “inhibiting the bromodomain-containing protein PCAF” includes inhibiting the expression and/or function of the bromodomain-protein PCAF.

The inhibitor may be of varied nature and origin including natural origin [e.g. plant, animal, eukaryotic, bacterial, viral] or synthetic [particularly an organic, inorganic, synthetic or semi-synthetic molecule]. For example it can be a nucleic acid, a polynucleotide, a polypeptide, a protein, a peptide or a chemical compound. In one aspect the inhibitor is selective for a particular bromodomain-containing protein with no activity against other bromodomain-containing proteins.

In one aspect the inhibitor is an antisense nucleic acid capable of inhibiting transcription of the bromodomain-containing proteins or translation of the corresponding messenger RNA. The antisense nucleic acid can comprise all or part of the sequence of the bromodomain-containing protein, or of a sequence that is complementary thereto. The antisense sequence can be a DNA, an RNA (e.g. siRNA), a ribozyme, etc. It may be single-stranded or double stranded. It can also be an RNA encoded by an antisense gene. When an antisense nucleic acid comprising part of the sequence of the gene or messenger RNA under consideration is being used, it is preferred to use a part comprising at least 10 consecutive bases from the sequence, more preferably at least 15, in order to ensure specific hybridisation. In the case of an antisense oligonucleotide, it typically comprises less than 100 bases, for example in the order of 10 to 50 bases. This oligonucleotide can be modified to improve its stability, its nuclease resistance, its cell penetration, etc. Perfect complementarily between the sequence of the antisense molecule and that of the target gene or messenger RNA is not required, but is generally preferred.

According to another embodiment, the inhibitor compound is a polypeptide. It may be, for example a peptide comprising a region of the bromodomain-containing protein, and capable of antagonising its activity. A peptide advantageously comprises from 5 to 50 consecutive amino acids of the primary sequence of the bromodomain-containing protein under consideration, typically from 7 to 40. The polypeptide can also be an antibody against the bromodomain-containing protein, or a fragment or derivative of such an antibody, for example a Fab fragment, a CDR region, or, more preferably, a single chain antibody (e.g. ScFv). Single chain antibodies are particularly advantageous insofar as they can act in a specific and intracellular fashion to modulate the activity of a target protein. Such antibodies, fragments, or derivatives can be produced by conventional techniques comprising immunising an animal and recovering the serum (polyclonal) or spleen cells (in order to produce hybridomas by fusion with appropriate cell lines).

Methods for producing polyclonal antibodies in various species are described in the prior art. Typically, the antigen is combined with an adjuvant (e.g. Freund's adjuvant) and administered to an animal, typically by subcutaneous injection. Repeated injections can be performed. Blood samples are collected and the immunoglobulin or serum is separated. Conventional methods for producing monoclonal antibodies comprise immunising of an animal with an antigen, followed by recovery of spleen cells, which are then fused with immortalised cells, such as myeloma cells. The resulting hybridomas produce monoclonal antibodies and can be selected by limiting dilution in order to isolate individual clones. Fab or F(ab′)2 fragments can be produced by protease digestion, according to conventional techniques.

According to another embodiment, the inhibitor is a chemical compound, of natural or synthetic origin, particularly an organic or inorganic molecule, capable of modulating the expression or the activity of the bromodomain-containing protein. In a particular embodiment, the inhibitor is a small molecule.

As used herein, the “effective amount” means that amount of a drug or pharmaceutical agent that will elicit the biological or medical response of a tissue, system, animal or human that is being sought, for instance, by a researcher or clinician. Furthermore, the term “therapeutic amount” means any amount which as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. “Therapy” and “treatment” may include treatment and/or prophylaxis.

While it is possible that, for use in therapy, the inhibitor may be administered as the raw chemical, it is possible to present the active ingredient (inhibitor) as a pharmaceutical composition. Accordingly, the invention further provides pharmaceutical compositions comprising an agent which inhibits the bromodomain-containing protein, PCAF, and one or more pharmaceutically acceptable carriers, diluents, or excipients. The carrier(s), diluents(s) or excipient(s) must be acceptable in the sense of being compatible with the other ingredients of the composition and not deleterious to the recipient thereof. In accordance with another aspect of the invention there is also provided a process for the preparation of a pharmaceutical composition including the agent, or pharmaceutically acceptable salts thereof, with one or more pharmaceutically acceptable carriers, diluents or excipients. The pharmaceutical composition can be for use in the treatment and/or prophylaxis of any of the conditions described herein.

Pharmaceutical compositions may be presented in unit dose forms containing a predetermined amount of agent per unit dose. Preferred unit dosage compositions are those containing a daily dose or sub-dose, or an appropriate fraction thereof, of an active ingredient. Such unit doses may therefore be administered once or more than once a day. Such pharmaceutical compositions may be prepared by any of the methods well known in the pharmacy art.

Pharmaceutical compositions may be adapted for administration by any appropriate route, for example by the oral (including buccal or sublingual), rectal, inhaled, intranasal, topical (including buccal, sublingual or transdermal), vaginal or parenteral (including subcutaneous, intramuscular, intravenous or intradermal) route. Such compositions may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the agent can be combined with an oral, non-toxic pharmaceutically acceptable inert carrier such as ethanol, glycerol, water and the like. Powders are prepared by reducing the compound to a suitable fine size and mixing with a similarly prepared pharmaceutical carrier such as an edible carbohydrate, as, for example, starch or mannitol. Flavouring, preservative, dispersing and colouring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, glidants, lubricants, sweetening agents, flavours, disintegrating agents and colouring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the agent. Syrups can be prepared by dissolving the compound in a suitably flavoured aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavour additive such as peppermint oil or natural sweeteners or saccharin or other artificial sweeteners, and the like can also be added.

Where appropriate, dosage unit compositions for oral administration can be microencapsulated. The composition can also be prepared to prolong or sustain the release as for example by coating or embedding particulate material in polymers, wax or the like.

The compounds of the invention may also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Pharmaceutical compositions adapted for transdermal administration may be presented as discrete patches intended to remain in intimate contact with the epidermis of the recipient for a prolonged period of time.

Pharmaceutical compositions adapted for topical administration may be formulated as ointments, creams, suspensions, lotions, powders, solutions, pastes, gels, sprays, aerosols or oils.

For treatments of the eye or other external tissues, for example mouth and skin, the compositions are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical compositions adapted for topical administrations to the eye include eye drops wherein the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or as enemas.

Dosage forms for nasal or inhaled administration may conveniently be formulated as aerosols, solutions, suspensions drops, gels or dry powders.

For compositions suitable and/or adapted for inhaled administration, it is preferred that the agent is in a particle-size-reduced form, and more preferably the size-reduced form is obtained or obtainable by micronisation. The preferable particle size of the size-reduced (e.g. micronised) compound or salt or solvate is defined by a D50 value of about 0.5 to about 10 microns (for example as measured using laser diffraction). Compositions adapted for administration by inhalation include the particle dusts or mists. Suitable compositions wherein the carrier is a liquid for administration as a nasal spray or drops include aqueous or oil solutions/suspensions of the active ingredient which may be generated by means of various types of metered dose pressurised aerosols, nebulizers or insufflators.

Aerosol formulations, e.g. for inhaled administration, can comprise a solution or fine suspension of the agent in a pharmaceutically acceptable aqueous or non-aqueous solvent. Aerosol formulations can be presented in single or multidose quantities in sterile form in a sealed container, which can take the form of a cartridge or refill for use with an atomising device or inhaler. Alternatively the sealed container may be a unitary dispensing device such as a single dose nasal inhaler or an aerosol dispenser fitted with a metering valve (metered dose inhaler) which is intended for disposal once the contents of the container have been exhausted.

Where the dosage form comprises an aerosol dispenser, it preferably contains a suitable propellant under pressure such as compressed air, carbon dioxide or an organic propellant such as a hydrofluorocarbon (HFC). Suitable HFC propellants include 1,1,1,2,3,3,3-heptafluoropropane and 1,1,1,2-tetrafluoroethane. The aerosol dosage forms can also take the form of a pump-atomiser. The pressurised aerosol may contain a solution or a suspension of the active compound. This may require the incorporation of additional excipients e.g. co-solvents and/or surfactants to improve the dispersion characteristics and homogeneity of suspension formulations. Solution formulations may also require the addition of co-solvents such as ethanol. Other excipient modifiers may also be incorporated to improve, for example, the stability and/or taste and/or fine particle mass characteristics (amount and/or profile) of the formulation.

For pharmaceutical compositions suitable and/or adapted for inhaled administration, the pharmaceutical composition may be a dry powder inhalable composition. Such a composition can comprise a powder base such as lactose, glucose, trehalose, mannitol or starch, the agent, (preferably in particle-size-reduced form, e.g. in micronised form), and optionally a performance modifier such as L-leucine or another amino acid, cellobiose octaacetate and/or metals salts of stearic acid such as magnesium or calcium stearate.

Aerosol formulations are preferably arranged so that each metered dose or “puff” of aerosol contains a particular amount of a compound of the invention. Administration may be once daily or several times daily, for example 2, 3 4 or 8 times, giving for example 1, 2 or 3 doses each time. The overall daily dose and the metered dose delivered by capsules and cartridges in an inhaler or insufflator will generally be double those with aerosol formulations.

Pharmaceutical compositions adapted for vaginal administration may be presented as pessaries, tampons, creams, gels, pastes, foams or spray formulations.

Pharmaceutical compositions adapted for parental administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the compositions may include other agents conventional in the art having regard to the type of formulation in question, for example those suitable for oral administration may include flavouring agents.

Antisense or RNA interference molecules may be administered to the mammal in need thereof. Alternatively, constructs including the same may be administered. Such molecules and constructs can be used to interfere with the expression of the protein of interest, e.g., the bromodomain-containing protein and as such, modify gene expression. Typically delivery is by means known in the art.

Antisense or RNA interference molecules can be delivered in vitro to cells or in vivo, e.g., to tumors of a mammal. Nodes of delivery can be used without limitations, including: intravenous, intramuscular, intraperitoneal, intra-arterial, local delivery during surgery, endoscopic, subcutaneous, and per os. Vectors can be selected for desirable properties for any particular application. Vectors can be viral or plasmid. Adenoviral vectors are useful in this regard. Tissue-specific, cell-type specific, or otherwise regulatable promoters can be used to control the transcription of the inhibitory polynucleotide molecules. Non-viral carriers such as liposomes or nanospheres can also be used.

A therapeutically effective amount of the agent will depend upon a number of factors including, for example, the age and weight of the subject, the precise condition requiring treatment and its severity, the nature of the formulation, and the route of administration, and will ultimately be at the discretion of the attendant physician or veterinarian. In particular, the subject to be treated is a mammal, particularly a human.

The agent may be administered in a daily dose. This amount may be given in a single dose per day or more usually in a number (such as two, three, four, five or six) of sub-doses per day such that the total daily dose is the same.

The agent may be employed alone or in combination with other therapeutic agents.

The agent for use in the present invention may be used in combination with or include one or more other therapeutic agents and may be administered either sequentially or simultaneously by any convenient route in separate or combined pharmaceutical compositions.

The agent and pharmaceutical compositions contain the invention may be used in combination with or include one or more other therapeutic agents, for example selected from NSAIDS, corticosteroids, COX-2 inhibitors, cytokine inhibitors, anti-TNF agents, inhibitors oncostatin M, anti-malarials, immunsuppressive and cytostatics.

Methods of Treatment and Diseases

Provided herein are methods of treatment or prevention of autoimmune and inflammatory conditions and diseases that can be improved by inhibiting the bromodomain-containing protein PCAF and thereby, e.g., modulate the level of expression of acetylation activated and acetylation repressed target genes. A method may comprise administering to a subject, e.g. a subject in need thereof, a therapeutically effective amount of an agent described herein.

Thus in one aspect there is provided the use of an inhibitor of the bromodomain-containing protein PCAF in the manufacture of a medicament for treating autoimmune and inflammatory diseases or conditions.

In a further aspect there is provided a method of treatment of autoimmune and inflammatory diseases or conditions in a mammal comprising administering a therapeutically effective amount of an inhibitor of the bromodomain-containing protein PCAF.

In one aspect the bromodomain-containing protein is PCAF.

In one aspect the inhibitor inhibits the bromodomain-containing protein PCAF.

Based at least on the fact that increased histone acetylation has been found to be associated with inflammation, a method for treating inflammation in a subject may comprise administering to the subject a therapeutically effective amount of one or more agents that decrease acetylation or restore acetylation to its level in corresponding normal cells, or prevent binding of proteins such as PCAF to acetylated histones.

Inflammation represents a group of vascular, cellular and neurological responses to trauma. Inflammation can be characterised as the movement of inflammatory cells such as monocytes, neutrophils and granulocytes into the tissues. This is usually associated with reduced endothelial barrier function and oedema into the tissues. Inflammation can be classified as either acute or chronic. Acute inflammation is the initial response of the body to harmful stimuli and is achieved by the increased movement of plasma and leukocytes from the blood into the injured tissues. A cascade of biochemical event propagates and matures the inflammatory response, involving the local vascular system, the immune system, and various cells within the injured tissue. Prolonged inflammation, known as chronic inflammation, leads to a progressive shift in the type of cells which are present at the site of inflammation and is characterised by simultaneous destruction and healing of the tissue from the inflammatory process.

When occurring as part of an immune response to infection or as an acute response to trauma, inflammation can be beneficial and is normally self-limiting. However, inflammation can be detrimental under various conditions. This includes the production of excessive inflammation in response to infectious agents, which can lead to significant organ damage and death (for example, in the setting of sepsis). Moreover, chronic inflammation is generally deleterious and is at the root of numerous chronic diseases, causing severe and irreversible damage to tissues. In such settings, the immune response is often directed against self-tissues (autoimmunity), although chronic responses to foreign entities can also lead to bystander damage to self tissues.

The aim of anti-inflammatory therapy is therefore to reduce this inflammation, to inhibit autoimmunity when present and to allow for the physiological process or healing and tissue repair to progress.

The agents may be used to treat inflammation of any tissue and organs of the body, including musculoskeletal inflammation, vascular inflammation, neural inflammation, digestive system inflammation, ocular inflammation, inflammation of the reproductive system, and other inflammation, as exemplified below.

Musculoskeletal inflammation refers to any inflammatory condition of the musculoskeletal system, particularly those conditions affecting skeletal joints, including joints of the hand, wrist, elbow, shoulder, jaw, spine, neck, hip, knew, ankle, and foot, and conditions affecting tissues connecting muscles to bones such as tendons. Examples of musculoskeletal inflammation which may be treated with compounds of the invention include arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic).

Ocular inflammation refers to inflammation of any structure of the eye, including the eye lids. Examples of ocular inflammation which may be treated with the compounds of the invention include blepharitis, blepharochalasis, conjunctivitis, dacryoadenitis, keratitis, keratoconjunctivitis sicca (dry eye), scleritis, trichiasis, and uveitis.

Examples of inflammation of the nervous system which may be treated with the compounds of the invention include encephalitis, Guillain-Barre syndrome, meningitis, neuromyotonia, narcolepsy, multiple sclerosis, myelitis and schizophrenia.

Examples of inflammation of the vasculature or lymphatic system which may be treated with the compounds of the invention include arthrosclerosis, arthritis, phlebitis, vasculitis, and lymphangitis.

Examples of inflammatory conditions of the digestive system which may be treated with the compounds of the invention include cholangitis, cholecystitis, enteritis, enterocolitis, gastritis, gastroenteritis, inflammatory bowel disease (such as Crohn's disease and ulcerative colitis), ileitis, and proctitis.

Examples of inflammatory conditions of the reproductive system which may be treated with the compounds of the invention include cervicitis, chorioamnionitis, endometritis, epididymitis, omphalitis, oophoritis, orchitis, salpingitis, tubo-ovarian abscess, urethritis, vaginitis, vulvitis, and vulvodynia.

The agents may be used to treat autoimmune conditions having an inflammatory component. Such conditions include acute disseminated alopecia universalise, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn's disease, diabetes mellitus type 1, giant cell arteritis, goodpasture's syndrome, Grave's disease, Guillain-Barre syndrome, Hashimoto's disease, Henoch-Schönlein purpura, Kawasaki's disease, lupus erythematosus, microscopic colitis, microscopic polyarteritis, mixed connective tissue disease, multiple sclerosis, myasthenia gravis, opsocionus myoclonus syndrome, optic neuritis, ord's thyroiditis, pemphigus, polyarteritis nodosa, polymyalgia, rheumatoid arthritis, Reiter's syndrome, Sjogren's syndrome, temporal arteritis, Wegener's granulomatosis, warm autoimmune haemolytic anemia, interstitial cystitis, lyme disease, morphea, psoriasis, sarcoidosis, scleroderma, ulcerative colitis, and vitiligo.

The agents may be used to treat T-cell mediated hypersensitivity diseases having an inflammatory component. Such conditions include contact hypersensitivity, contact dermatitis (including that due to poison ivy), uticaria, skin allergies, respiratory allergies (hayfever, allergic rhinitis) and gluten-sensitive enteropathy (Celliac disease).

Other inflammatory conditions which may be treated with the agents include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, percarditis, peritonoitis, pharyngitis, pleuritis, pneumonitis, prostatistis, pyelonephritis, and stomatisi, transplant rejection (involving organs such as kidney, liver, heart, lung, pancreas (e.g., islet cells), bone marrow, cornea, small bowel, skin allografts, skin homografts, and heart valve xengrafts, serum sickness, and graft vs host disease), acute pancreatitis, chronic pancreatitis, acute respiratory distress syndrome, Sexary's syndrome, congenital adrenal hyperplasis, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, astopic dermatitis, drug hypersensistivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenic purpura in adults, secondary thrombocytopenia in adults, acquired (autoimmune) haemolytic anemia, leukaemia and lymphomas in adults, acute leukaemia of childhood, regional enteritis, autoimmune vasculitis, multiple sclerosis, chronic obstructive pulmonary disease, solid organ transplant rejection, sepsis. Preferred treatments include treatment of transplant rejection, rheumatoid arthritis, psoriatic arthritis, multiple sclerosis, Type 1 diabetes, asthma, inflammatory bowel disease, systemic lupus erythematosis, psoriasis, chronic pulmonary disease, and inflammation accompanying infectious conditions (e.g., sepsis).

The methods of treatment and uses of the invention can be used in mammals, particularly in humans.

The present invention also provides a method for identifying agents which may be candidate compounds for the treatment of autoimmune and inflammatory diseases or conditions comprising determining whether a compound is capable of inhibiting the bromodomain-containing protein, PCAF.

Screening Methods

The present invention proposes, for the first time that the bromodomain-containing PCAF, is a potential therapeutic target for the treatment of autoimmune and inflammatory diseases and conditions and/or cancer. Thus, the present invention provides a new target for the identification, validation, selection and optimisation of active agents on the basis of their ability to modulate the expression or activity of the bromodomain-containing protein PCAF. Such active agents include inhibitors as described above.

The present invention thus pertains to a method of identifying, screening, characterising or defining an agent which is capable of modulating the activity of the bromodomain-containing protein PCAF. The methods can be used for screening for example large numbers of candidate compounds for clinical use in inflammatory and autoimmune diseases or cancer.

The assays (screening methods) may be performed in a cell-based system, an animal system or by a cell free system. Such techniques will be apparent to a person skilled in the art and may be based on a measure of interaction [e.g. binding, displacement or competition assays) or a measure of a function of activity, transcription and the like.

Thus, for example, the present invention provides a method of testing the ability of an agent to modulate the expression of the bromodomain-containing protein PCAF, particularly to inhibit expression. In another example the present invention provides a method of testing the ability of an agent to bind to and optionally modulate the activity of the bromodomain-containing protein PCAF, particularly to inhibit activity. In a further example the present invention provides a method for testing the ability of an agent to modulate the activity of the bromodomain-containing protein PCAF, particularly to inhibit activity.

Provided herein are screening methods for identifying agents that inhibit bromodomain-containing proteins as being potentially useful in the treatment of prevention of autoimmune and inflammatory diseases and conditions and/or cancer. One method involves screening for an inhibitor of bromodomain-containing protein activity, including the steps of contacting a peptide, which may be modified by acetylation, with a bromodomain, particularly the bromodomain-containing protein PCAF or a fragment thereof in the presence and in the absence of a test substance, and identifying a test substance as an inhibitor or activator of bromodomain activity. Test agents (or substances) for screening as inhibitors of the bromodomain can be from any source known in the art. They can be natural products, purified or mixtures, synthetic compounds, members of compound libraries, etc. The test substances can be selected from those that have been identified previously to have biological or drug activity or from those that have not.

In a further aspect the method of screening for an inhibitor of bromodomain-containing protein includes a binding assay. Thus a compound which inhibits the binding of the bromodomain-containing protein PCAF to its substrate can be identified in competition or direct binding assays. Both direct and competition binding assay formats are similar to the formats used in immunoassays and receptor binding assays and will be generally known to a person skilled in the art.

In one aspect the bromodomain-containing protein is PCAF.

Typically the methods use peptides of the PCAF protein. In particular the methods use a human protein. More particularly the protein has the amino acid sequence of human PCAF protein as described in accession number NP_{—}003875.3 or a fragment thereof or a sequence having at least 75%, 80%, 85%, 90%, 91%, 92%, 93%, 94%, 95%, 96%, 97%, 98% or 99% homology to the amino acid sequence of human PCAF protein or a fragment thereof.

Preferably the fragments are at least 110 amino acids long and include the bromodomain.

The present invention further contemplates analogues of the amino acid sequences formed by conservative amino acid substitution. The principle behind conservative amino acid substitution is that certain amino acid pairs have compatible side chains such that, when one amino acid is substituted for the other, there will be only minimal changes in the tertiary structure of the peptide. Rules for conservative substitutions are explained in Bowie et al. Science 247(1990) 1306-1310. It is an object of the present invention to utilise polypeptides, fragments and variants that retain the ability of the protein to bind substrate. I

Where required, each of the polypeptides, fragments and variants, where required, may be provided either in purified or un-purified form, for example as cellular extracts or by purification of the relevant component from such extracts. Alternatively, the polypeptides, fragments and variants can be recombinantly produced by recombinant expression techniques, and purified for use in the assay. Alternatively, the polypeptides, fragments and variants can be expressed recombinantly in a cell for use in cell based assays.

Typically, a polynucleotide encoding the relevant component is provided within an expression vector. Such expression vectors are routinely constructed in the art and may for example involve the use of plasmid DNA and appropriate initiators, promoters, enhancers and other elements, such as for example polyadenylation signals which may be necessary and which are positioned in the correct orientation in order to allow full protein expression. Suitable vectors would be very readily apparent to those of skill in the art, such as those described in more detail in the examples of the present application. Promoter sequences may be inducible or constitutive promoters depending on the selected assay format.

As natural substrates for PCAF have been described to include histone 3 acetylated on lysine 36 (H3K36Ac), H3K9Ac, H4K8Ac, H3K14Ac, H4K16Ac H4K20Ac and H4R3me2, as well as the Tat peptide of human immunodeficiency virus-1 acetylated on lysine 50 (TatK50Ac) (4-7), preferred substrates could comprise peptides corresponding to these sequences and modifications. Conversely, other peptides with suitable affinity for PCAF could be utilised.

Thus for example the substrate may be a peptide, such a synthetic peptide comprising N-terminal residues of histone 3 or 4, at least 10, 20, 30, 40, 50 or full length. The substrate may be at least 70%, 75%, 80%, 85%, 90%, 91%, 92%, 93%, 94%, 95%, 96%, 97%, 98%, 99%, homologous thereto.

It may be also preferred to use a substrate selected from bulk histones, synthetic peptides and nucleosomes.

The following examples are set forth to illustrate the effectiveness of the approach described in the present invention and to further exemplify particular applications of general processes described above. Accordingly, the following Example section is in no way intended to limit the scope of the invention contemplated herein.

### EXAMPLES

To investigate whether bromodomain-containing proteins might represent targets for treatment of autoimmunity and inflammatory diseases, we screened an siRNA library targeting 40 bromodomain-containing proteins (Table 1) for effects on human dendritic cell function. siRNAs bind specifically to an mRNA transcript that bears a complementary nucleotide sequence and subsequently reduces expression of the protein encoded by that specific mRNA. Since a number of disparate factors can influence the whether an siRNA can effectively reduce expression of its target gene, and advanced algorithms capable of accurately predicting efficacious siRNAs have not yet been developed, it has been recommended to use a minimum of three to four siRNAs against a given target when conducting a screen (8). We used at least four distinct siRNAs targeting each gene, including PCAF (Table 2) for our studies.

TABLE 1
List of bromodomain-containing protein targets in library

ATAD2
ATAD2B
BAZ1A
BAZ1B
BAZ2A
BAZ2B
BRD1
BRD2
BRD3
BRD4
BRD7
BRD8
BRD9
BRDT
BRPF1
BRPF3
BRWD1
BRWD3
CECR2
CREBBP
EP300
FALZ
KAT2A
KAT2B (PCAF)
PBRM1
PHIP
PRKCBP1
SMARCA2
SMARCA4
SP100
SP110
SP140
SP140L
TAF1
TAF1L
TRIM24
TRIM28
TRIM33
TRIM66
PCAF

TABLE 2
List of PCAF siRNA target sequences

| siRNA name | Target sequence |
|---|---|
| PCAF_6: | CGGAGTGTACTCCGCCTGCAA |
| PCAF_7: | CAGCAAATAATTGTCAGTCTA |
| PCAF_9: | ACAGTCTACCTCGGTACGAAA |
| PCAF_10: | ATCGCCGTGAAGAAAGCGCAA |

We tested the effect of introducing these siRNAs into primary monocyte-derived human dendritic cells. Dendritic cells are thought to make major contributions to autoimmunity and inflammation through their ability to present antigens and activate T cells and through their production of pro-inflammatory cytokines. Conversely, these cells can also produce anti-inflammatory cytokines which counteract inflammatory processes. Hence, we assessed the effect of siRNAs targeting bromodomain-containing proteins on the production of a variety of cytokines, including the pro-inflammatory cytokine TNF-α, which has a central role in autoimmune and inflammatory disease (9), and the anti-inflammatory cytokine IL-10, which has a crucial function in inhibiting inflammation (10).

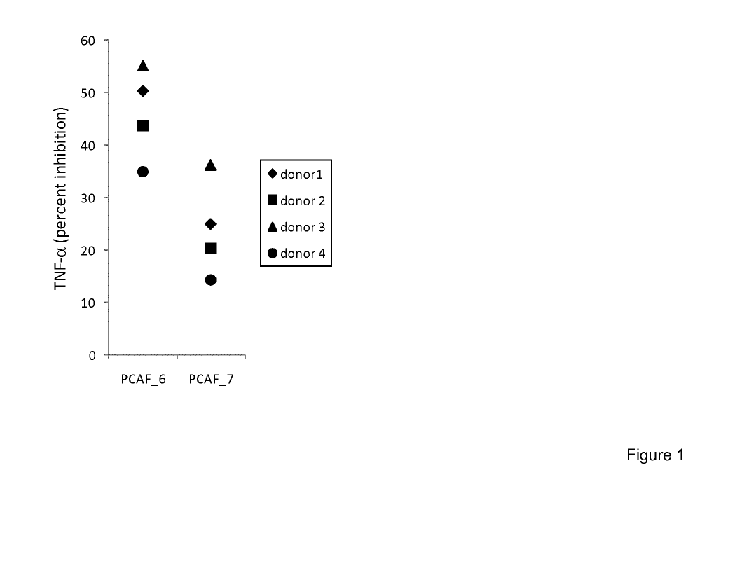

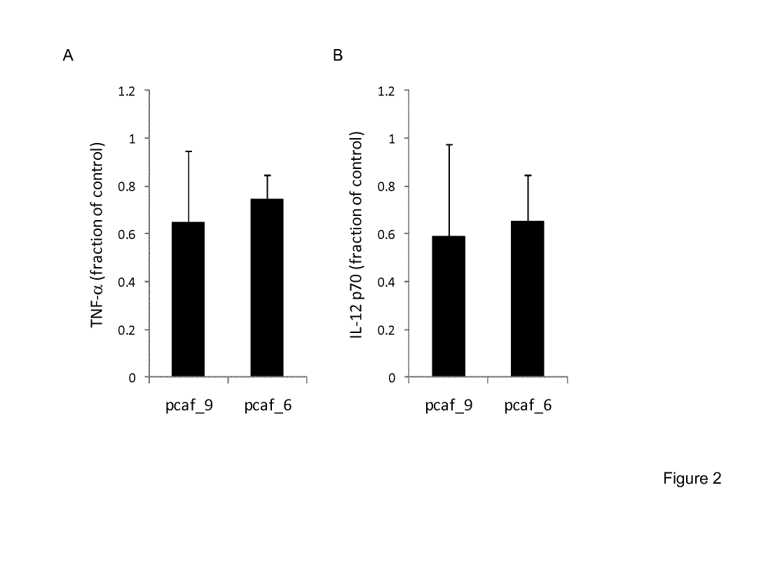

The siRNAs were introduced into dendritic cells by transfection, after which the cells were stimulated by treatment with lipopolysaccharide (LPS), a bacterial component. The quantities of cytokines present in the medium approximately twenty-four hours after activation were measured. As shown in FIG. 1, two distinct siRNAs targeting the bromodomain-containing protein, PCAF, were found to inhibit TNF-α production by dendritic cells. To confirm the results from the siRNA library screen, we further assessed the role of PCAF in dendritic cell cytokine production by comparing various PCAF-targeting siRNAs with scrambled control siRNA for effects on dendritic cell cytokine production. As shown in FIG. 2, we found that an additional PCAF-targeting siRNA also inhibited LPS-induced TNF-α production. Moreover, PCAF siRNAs were also found to inhibit dendritic cell production of IL12p70, another pro-inflammatory cytokine implicated in inflammatory/autoimmune disease (11,12). Hence, these results suggested that PCAF contributes to the production of pro-inflammatory cytokines by dendritic cells.

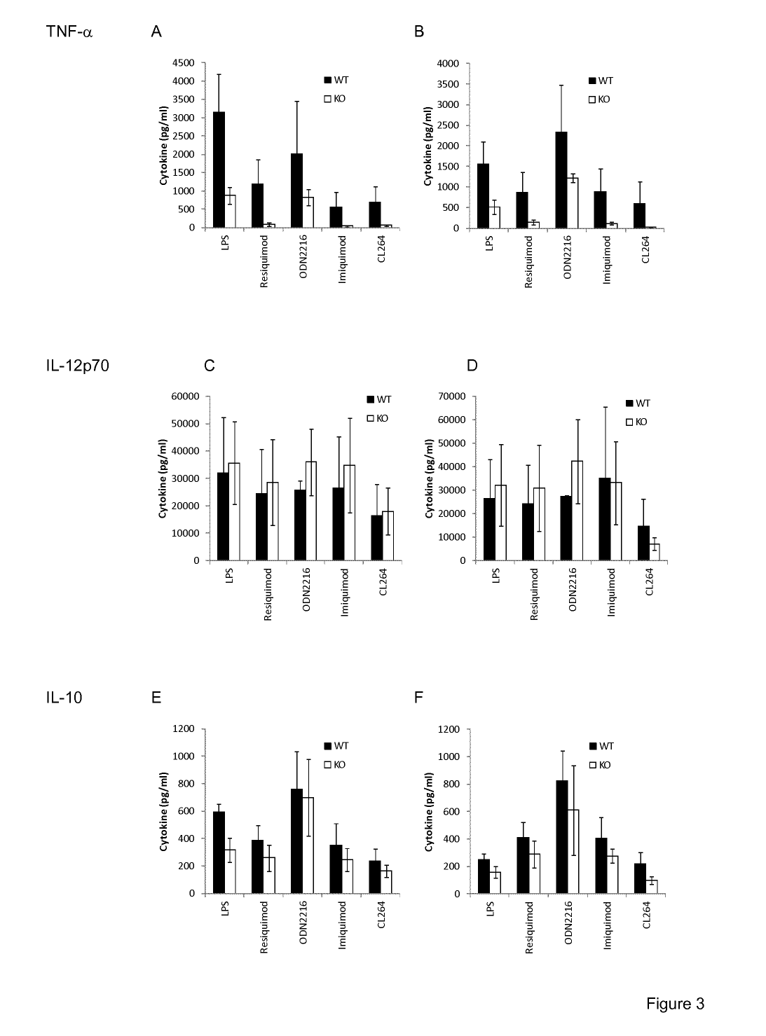

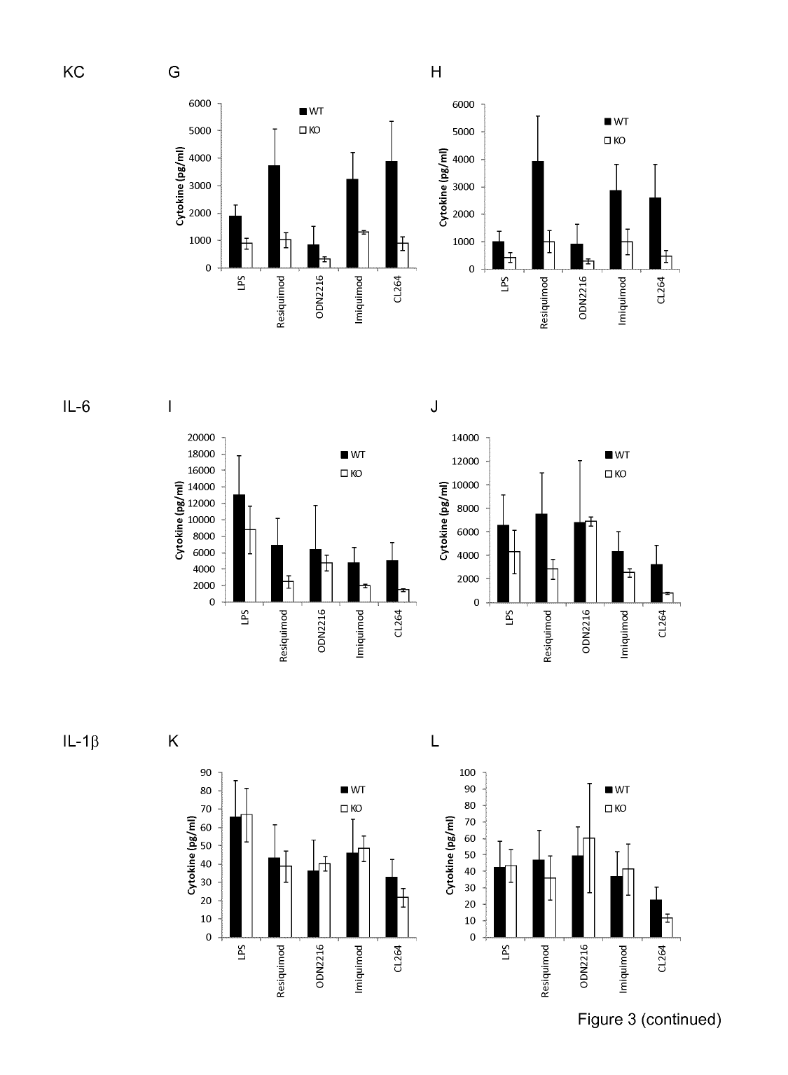

The functional role of PCAF in inflammatory cytokine production was investigated further by comparing cytokine production by dendritic cells from normal (wild-type; WT) mice with those of dendritic cells from mice in which the PCAF gene has been functionally deleted (PCAF knockout mice; KO) (FIG. 3). In addition to LPS, the cells were treated with several other pro-inflammatory stimuli: resiquimod (an agonist of TLR7 and TLR8), ODN2216 (a TLR9 agonist), imiquimod (a TLR7 agonist) and CL264 (a TLR7 agonist). Two different doses of each stimulus were used. The mice are referenced in 14 and supplied by INSERM. (INSERM U896, 34298 Cedex 5 Montpellier).

In accordance with the results observed after siRNA treatment of human dendritic cells, PCAF KO mouse dendritic cells secreted greatly reduced quantities of TNF-α compared to WT dendritic cells after LPS stimulation (FIGS. 3A, B). TNF-α secretion was also reduced in PCAF KO dendritic cells treated with each of the other stimuli. This did not reflect a global defect in cytokine production by the PCAF KO cells, since production of IL-12p70, IL-10 and IL-1β was very similar between WT and PCAF KO cells. However, PCAF KO dendritic cells did show reduced production of some additional pro-inflammatory cytokines compared to WT cells. Thus, production of KC (CXCL1, the mouse equivalent of human IL-8) was lower in PCAF KO than WT dendritic cells treated with all 5 stimuli (FIGS. 3G, H), while the amount of IL-6 secreted by PCAF KO dendritic cells was decreased for all stimuli except ODN2216 (FIGS. 3I, J).

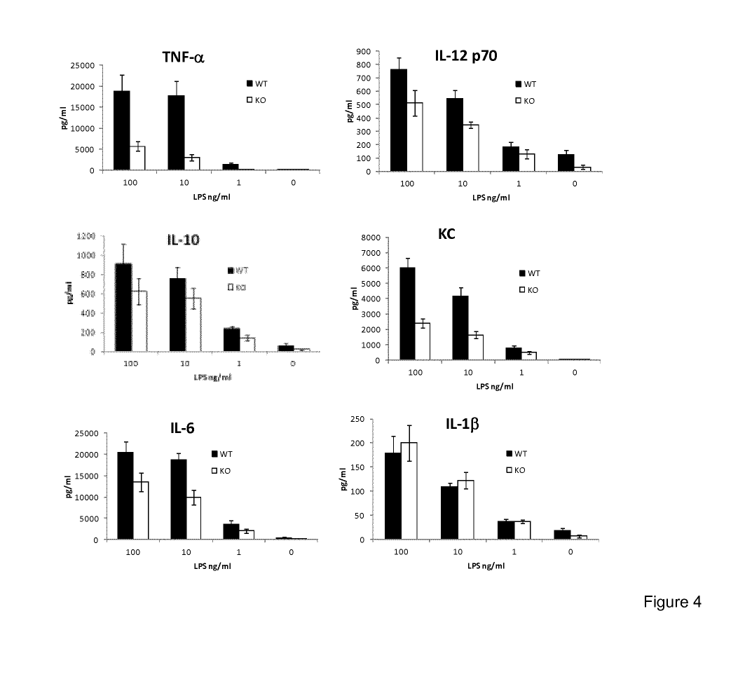

We also examined the effect of PCAF deficiency on another cell type involved in the production of inflammatory cytokines, namely the macrophage. Macrophages from WT and PCAF KO mice were treated with different concentrations of LPS and the secretion of various cytokines measured (FIG. 4). As for dendritic cells, PCAF KO macrophages produced much lower amounts of TNF-α than WT macrophages at all doses of LPS tested (FIG. 4A). Also similar to the results with dendritic cells, the amounts of KC and IL-6 secreted by PCAF KO macrophages were strongly reduced compared to WT cells. Production of IL-12p70 and IL-10 was slightly reduced in PCAF KO macrophages compared to WT macrophages at some concentrations of LPS, while LPS-induced secretion of 1L-1β did not differ between the two.

Taken together, these results provide evidence that PCAF makes an important contribution to the production of pro-inflammatory cytokines in response to multiple activating stimuli in key immunological cell types, dendritic cells and macrophages. Moreover, a paper published after submission of this patent application has provided independent confirmation that cells from PCAF KO mice show reduced expression of a number of pro-inflammatory genes (13). Therefore, we propose that approaches to inhibit the expression and/or function of this bromodomain-containing protein would be of benefit for the treatment of autoimmunity and inflammatory diseases and conditions.

Methods:

Human dendritic cell preparation: 15 ml Lymphoprep (Axis-Sheild PoC) was placed in 50 ml Accuspin tubes and the tubes spun at 2000 rpm for 1 min to allow the Lymphoprep to pass through the filter to the bottom of the tube. 200 ml of heparinsed human blood was obtained from healthy donors and divided between 8×Accuspin/Lymphoprep tubes. The blood samples were then spun at 2500 rpm for 20 min without brake. The top layer was transfered to a new falcon tube, topped up with PBS and centrifuged at 1600 rpm for 10 min. Cell pellets were pooled together, PBS was added to 45 ml, and samples were centrifuged at 1500 rpm for 5 min. Cells were re-suspended in 10 ml MACS buffer and spun down (15000 rpm, 5 mins with brake). Cells were re-suspended in a total of 80 μl/10⁷ cells ice cold Miltenyi buffer, to which was added MACS CD14 beads (20 μl/10⁷ cells). Cells were incubated at in the cold for 15 minutes, after which they were washed with cold Miltenyi buffer and centrifuged for 5 min at 1500 rpm. Pellets were re-suspended in Miltenyi buffer at 500 μl/10⁸ cells. Cell suspension was added to MACS Miltenyi separation columns (1 ml per column). Columns were washed with 3×3 ml 4° C. Miltenyi buffer. Bound CD14⁺ cells were subsequently eluted by removing columns from their magnetic holder, placing them over a 15 ml Falcon tube, and plunging 5 ml Miltenyi buffer through the columns using a supplied syringe barrel (Miltenyi Biotec). Eluted cell suspensions were spun down at 400×g (RT) for 10 min, resuspended in culture medium (RPMI 1640 medium (Invitrogen) supplemented with 2 mM L-glutamine (Invitrogen), 100 units/ml penicillin (Invitrogen) and 100 μg/ml streptomycin (Invitrogen)) with 5% foetal bovine serum (FBS, heat-inactivated, Invitrogen) and counted using a haemocytometer. The purified monocytes were resuspended at 5×10⁵ cells/ml in RPMI 1640/L-Glu (+10% FCS, pen/strep and glutamine) containing 30 ng/ml GMCSF and 20 ng/ml IL-4 and cultured for 6-7 days.

Human dendritic cell transfection and stimulation: siRNAs were transfected into dendritic cells using the Amaxa human monocyte 96 well nucleofector kit (Lonza VHPA-2007). 50,000 cells were re-suspended in 20 μl amaxa buffer, after which siRNAs were added to achieve a final concentration of 2 μM. The plate was placed into the Amaxa nucleofector and the monocyte program EA-100 applied to all wells. 100 μl of medium was added to each well of the Amaxa plate, and cells were immediately removed from the Amaxa plate wells (100 μl was removed from the plate to ensure a consistent volume recovered) and added to a second U′bottom plate containing an additional 100 μl of pre-warmed media. After incubation for 24-48 hours at 37° C., LPS was added at 100 ng/ml. Supernatants were collected 6-24 hours later and cytokines were measured using MSD plates and read in a MSD Sector 6000 plate reader. Cell viability was measured using the CellTiter-Glo® luminescent cell viability assay (Promega G7571) or the Cell Titre Blue cell viability assay reagent (Promega G8081).

Materials
Ficoll paque plus buffer (GE HealthCare Bio-Sciences cat#17-1440-03) (Histopaque)
Miltenyi buffer: PBS w/o Ca and Mg, 0.5% BSA, 2 mM EDTA
Miltenyi (MACS) CD14 beads: CD14 microbeads, human 2 mL, contains 0.1% BSA, 0.05% Azide
BSA: BSA solution 35% (Sigma cat#A7409-50ML)
EDTA: Ethylenediaminetetraacetic acid (Sigma cat#E7889-100ML)
Accuspin tubes (Sigma cat#A2055-10EA)
1×PBS without Ca and Mg, Invitrogen, Gibco, #14190-094
MACS Miltenyi separation columns—130-042-401
RPMI+10% FCS, 1% glutamine, 1% pen/strep
siRNAs: All siRNAs were obtained from Qiagen,
IL-4: (R&D systems #204-IL)

Generation and stimulation of mouse bone marrow-derived dendritic cells: Bone Marrow cells were isolated from severed mouse femurs and tibiae by flushing the bone marrow with RPMI using small gauge needles (G22-G26). After vigorous pipetting, single cell suspensions (total cell population) were passed through a 40 um cell strainer (Fisher, 22363547) into 15 mL conical tube. Cells were counted using the Sysmex-XT2000i (mouse WBC count used). Total cells were centrifuged at 300 g for 5 minutes, 4° C. and resuspended in media (RPMI+10% FBS+L-glutamine+Penicillin-Streptomycin+50 uM β-mercaptoethanol) to a density of 1×10⁶ cells/ml in media supplemented with 25 ng/ml mouse GM-CSF (PMC2016, Invitrogen). Bone marrow-derived dendritic cells were differentiated in sterile bacterial Petri dishes (1×10⁷ cells, 10 mL per dish).

After 7 days of differentiation, dendritic cells were counted using the Sysmex. Cells from each mouse were kept separated or pooled when necessary (according to phenotype) prior to stimulation. Cells were seeded in 96 flat-well plates at 2×10⁵ cells per well (100 ul per well) in complete RPMI and stimulated with 10 ul of the following TLRs ligands: LPS (Sigma, L4391-IMG, 100 ng/ml or 10 ng/ml final concentrations), Resiquimod (10 uM or 5 uM); ODN2216 (10 uM or 5 uM), Imiquimod (40 uM or 20 uM), and CL-264 (40 uM or 20 uM). Supernatants were collected 6 and 24 hours post stimulation and stored at −20° C. prior to quantification. Cytokines were measured using MSD plates and read in a MSD Sector 6000 plate reader.

Generation and stimulation of mouse bone marrow-derived macrophages: Single cell suspensions were generated from total bone marrow cells isolated as above and were sieved through 70 um cell strainers and counted with ViCell. Cell were resuspended at 5×10⁶/ml in complete DMEM (10% FBS+L-glutamine+Penicillin-Streptomycin+50 uM β-mercaptoethanol, NEAA, Sodium Pyruvate) supplemented with 5 ng/ml mouse M-CSF (Peprotech 315-02) and 5 ng/ml mouse IL3 (Peprotech 213-13). Cells were incubated for 24 hr in sterile bacterial Petri dishes (5×10⁷ cells, 10 ml per dish). Cells suspensions were transferred to new Petri dishes and incubated for further 6 days and then washed with PBS+5 mM EDTA+2% BSA for 15 minutes. Cells were removed from plastic, spun at 300 g for 5 minutes, washed with medium, and plated in 96 flat-well plates (2×10⁵ cells/well in 200 ul). Cells were activated with 22 ul LPS (100, 10, 1 ng/ml final concentration; Sigma E. coli LPS 0111:B4) for 6 hr. Supernatant was collected and stored at −20 C prior to analysis. Cytokines were measured using MSD plates and read in a MSD Sector 6000 plate reader.

### REFERENCES

(1) Struhl K. Histone acetylation and transcriptional regulatory mechanisms. Genes Dev. 1998 Mar. 1; 12(5):599-606.
(2) Jeanmougin F, Wurtz J M, Le Douarin B, Chambon P, Losson R. The bromodomain revisited. Trends Biochem Sci. 1997 May; 22(5):151-3.
(3) Tamkun J W, Deuring R, Scott M P, Kissinger M, Pattatucci A M, Kaufman T C, Kennison J A. Brahma: a regulator of Drosophila homeotic genes structurally related to the yeast transcriptional activator SNF2/SWI2. Cell. 1992 Feb. 7; 68(3):561-72.
(4) Dhalluin C, Carlson J E, Zeng L, He C, Aggarwal A K, Zhou M M. Structure and ligand of a histone acetyltransferases bromodomain. Nature. 1999 Jun. 3; 399(6735):491-6.
(5) Mujtaba S, He Y, Zeng L, Farooq A, Carlson J E, Ott M, Verdin E, Zhou M M. Structural basis of lysine-acetylated HIV-1 Tat recognition by PCAF bromodomain. Mol Cell. 2002 March; 9(3):575-86.
(6) Zeng L, Zhang Q, Gerona-Navarro G, Moshkina N, Zhou M M. Structural basis of site-specific histone recognition by the bromodomains of human coactivators PCAF and CBP/p300. Structure. 2008 April; 16(4):643-52.
(7) Li X, Hu X, Patel B, Zhou Z, Liang S, Ybarra R, Qiu Y, Felsenfeld G, Bungert J, Huang S. H4R3 methylation facilitates beta-globin transcription by regulating histone acetyltransferase binding and H3 acetylation. Blood. 2010 Mar. 11; 115(10):2028-37. Epub 2010 Jan. 12.
(8) Wolters N M, MacKeigan J P. From sequence to function: using RNAi to elucidate mechanisms of human disease. Cell Death Differ. 2008 May; 15(5):809-19.
(9) Sethi G, Sung B, Kunnumakkara A B, Aggarwal B B. Targeting TNF for Treatment of Cancer and Autoimmunity. Adv Exp Med Biol. 2009; 647:37-51.
(10) Mosser, D M, Zhang, X. Interleukin-10: new perspectives on an old cytokine. Immunol Rev. 2008; 226: 205-218.
(11) Peluso, I, Pallone, F, Monteleone, G. Interleukin-12 and Th1 immune response in Crohn's disease: pathogenetic relevance and therapeutic implication. World J Gastroenterol. 2006, 12: 5606-5610.
(12) Paunovic, P, Carroll, H P, Vandenbroeck, K, Gadina, M. Crossed signals: the role of interleukin (IL)-12, -17, -23 and -27 in autoimmunity. Rheumatology 2008, 47: 771-776.
(13) Bastiaansen, A J, Ewing, M M, de Boer, H C, van der Pouw Kraan, T C, de Vries, M R, Peters, E A, Welten, S M, Arens, R, Moore, S M, Faber, J E, Jukema, J W, Hamming, J F, Nossent, A Y, Quax, P H. Lysine Acetyltransferase PCAF Is a Key Regulator of Arteriogenesis. Arterioscler Thromb Vasc Biol. 2013 August; 33(8):1902-1910. Epub 2013 Jun. 20.
(14) Duclot F, Jacquet C, Gongora C, Maurice T. Alteration of working memory but not in anxiety or stress response in p300/CBP associated factor (PCAF) histone acetylase knockout mice bred on a C57BL/6 background. Neurosci Lett. 2010 May 21; 475(3):179-83.

1. A method of treating autoimmune and inflammatory diseases and conditions or cancer which comprises inhibiting the bromodomain-containing protein PCAF in a mammal.

2. A method of treating autoimmune and inflammatory diseases or conditions or cancer in a mammal, such as a human, which comprises the administration of a therapeutically effective amount of an inhibitor of the bromodomain-containing protein PCAF.

3. Use of an inhibitor of the bromodomain-containing protein PCAF in the manufacture of a medicament for the treatment of autoimmune and inflammatory diseases or conditions or cancer.

4. An inhibitor of the bromodomain-containing protein PCAF for use in the treatment of autoimmune and inflammatory diseases and conditions or cancer.

5. A pharmaceutical formulation for use in the treatment of autoimmune and inflammatory diseases or conditions or cancer, comprising an inhibitor of the bromodomain-containing protein PCAF, together with at least one pharmaceutical carrier.

6. A method for identifying compounds that will be useful in treating autoimmune and inflammatory diseases or conditions or cancer comprising the step of determining whether the compound inhibits or the step of determining whether the compound activates the bromodomain-containing protein PCAF.


SEQUENCE LISTING (4 sequences)

SEQ ID NO: 1 - DNA, 21 bp, Human
cggagtgtactccgcctgcaa

SEQ ID NO: 2 - DNA, 21 bp, Human
cagcaaataattgtcagtcta

SEQ ID NO: 3 - DNA, 21 bp, Human
acagtctacctcggtacgaaa

SEQ ID NO: 4 - DNA, 21 bp, Human
atcgccgtgaagaaagcgcaa